(19) United States
(12) Patent Application Publication
(10) Pub. No.: US 2016/0369065 A1
(43) Pub. Date: Dec. 22, 2016

(54) Functional Nanoparticle Composite Comprising Chitosan

(71) Applicants: Sivarooban Theivendran, Reno, NV (US); James J. Kubinec, Reno, NV (US); Terryll Riley Smith, Reno, NV (US)

(72) Inventors: Sivarooban Theivendran, Reno, NV (US); James J. Kubinec, Reno, NV (US); Terryll Riley Smith, Reno, NV (US)

(73) Assignee: Water Security Corporation, Sparks, NV (US)

(21) Appl. No.: 15/187,766

(22) Filed: Jun. 20, 2016

Related U.S. Application Data
(60) Provisional application No. 62/181,509, filed on Jun. 18, 2015.

(51) Int. Cl. C08J 3/075; A01N 43/16; A23L 3/3562; A61K 31/722; C08L 5/08; A61K 9/14; A01N 25/04

(52) U.S. Cl. CPC C08J 3/075; A61K 9/141; A61K 9/145; A61K 9/146; A61K 9/143; A01N 43/16; A01N 25/04; A61K 31/722; C08L 5/08; A23L 3/3562; C08J 2305/08; C08L 2201/54; C08L 2203/02; A23V 2002/00

(57) ABSTRACT

Methods for producing economical antimicrobial and antifungal nanoparticle composites are presented. The method includes converting chitosan or derivative thereof to nanoparticles by ionotropic gelation process and the nanoparticle composite in the presence of a process solution without any separation process to isolate the nanoparticles prior to application. The composition containing chitosan nanoparticles, ionotropic gelation agent, a nonionic surfactant, and an active halogen provides improved disinfectant and fungicidal properties against bacterial, viral and fungal contaminants along with prolonged residual effects. Methods and systems for decontamination, wound healing and plant growth enhancements are also presented.

### CROSS REFERENCE TO RELATED APPLICATIONS

This application claims priority to U.S. Provisional application No 62/181,509, filed Jun. 18, 2015

### FIELD OF TECHNOLOGY

The present disclosure relates to methods for producing economical functional nanoparticle composites. The nanoparticle composites may be incorporated into nontoxic household cleaners, would healing agents and agricultural growth promotors.

### BACKGROUND

The present invention relates to aqueous antimicrobial and antifungal compositions. In particular, the composition incorporates nontoxic nanoparticles for improved disinfectant and fungicidal properties. There is a growing need for effective, nontoxic decontaminating agents for eliminating biological contaminants from various surfaces including skin, food, agricultural, industrial and household utilities.

There are number of chemical and natural methods used for household cleaning purposes. Most of these applications have limitations in their activity or require high concentrations for effective performance against biological contaminants. The concentrated chemical usage for surface cleaning may cause severe health problems and require high volume wash water. There is a demand for natural, nontoxic cleaning products with effective residual functionalities. In general an effective delivery system is critical for the optimal function of natural cleaning ingredients due to their complex structural formation. Achieving effective disinfecting performance of natural ingredients through environmental friendly economical delivery systems for removal of biological contaminants is highly desirable.

Nano-scale particle delivery systems are an ideal candidate for surface cleaning in that they can provide uniform applications over extended surface area with residual effects using minimal ingredients. The nanoparticles (NPs) can selectively scavenge nano-structures of the biological contaminants which can dramatically improve the antimicrobial and antifungal efficacy.

Chitosan is a natural, biodegradable, nontoxic polymer with polycationic properties. Chitosan widely used for industrial applications such as biomedical, biotechnology, water treatment, food and agriculture due to its biocompatibility, biodegradability and bioactivity. A number of prior arts have demonstrated the use of chitosan in surface decontamination, wound healing, and plant growth enhancement applications. Unfortunately most these treatments fail to provide ultimate performance of the chitosan due to limited surface access, lack of consistent activities, and high production cost for defined constituents.

Chitosan has been demonstrated to be effective for antimicrobial and antifungal properties. For example, in U.S. Pat. No. 6,849,586 B2 disclosures, the hard surface cleaning compositions containing chitosan and surfactants in an acidic medium had demonstrable residual effects against soil, bacteria, and mold and biofilm formation. The U.S. Pat. No. 7,244,700 B2 describes the use of chitosan salt, chlorine, and a surfactant in an aqueous solution for antifungal activities. In both applications, the soluble chitosan is used as cationic polymer with antimicrobial or antifungal properties in combination with other chemical ingredients. Some of those previous formulations contain additive toxic chemicals with chitosan. In comparison, the use of chitosan nanoparticles in a nontoxic process solution is more desirable for surface decontamination applications due to its improved size dependent activities and effective production cost.

The U.S. Pat. No. 4,275,194 A describes the properties of chitosan-iodine adduct for wide range of surface disinfectant applications. Chitosan and its derivatives have also been demonstrated for effective use of treating or preventing various types of wound infections. When using chitosan for wound dressing, the chitosan dressing must be uniformly adherent on the wound surface without forming a fluid pocket. The chitosan dressing must be permeable in order to allow the water to evaporate. There are various methods demonstrating the use of chitosan dressing to combat wound infections. However, using an aerosol spray containing nanoparticles of chitosan and its derivatives in a nontoxic process solution can be more effective and desirable for controlling wound infections.

Chitosan and its derivatives are widely used in agricultural as growth promoting agents, and have been shown to improve plant defense mechanisms through cellular responses. The U.S. Pat. No. 8,946,119 B2 outlines the method of enhancing soy bean growth using chitosan oligosaccharide. The size dependent activity of chitosan is critical for agricultural applications. The challenge exists in preparing defined size chitosan nanoparticles via chemical methods. It is desirable to have defined, properly engineered chitosan nanoparticles in the presence of a nontoxic process solution in order to provide greater benefits in terms of production cost, production volume and effectiveness for high volume agricultural applications.

The availability of chitosan cationic charge sites are important for its functional properties. The optimal chitosan performance can be achieved when nano-scale particles are incorporated through effective delivery systems. The chitosan nanoparticles can be prepared by ionotropic gelation process. This widely used method utilizes low concentrations of gelation agent in an acidic medium followed by a nanoparticle separation process. Nanoparticle separation is the most labor intensive, time consuming and expensive part of the process in an industrial setting. Typical nanoparticle preparatory methods include a high speed centrifugation for a specified time period, followed by a physical removal of supernatant or process solution. The alternative membrane filtration technique for nanoparticle separation has limitations due to size dependent activities. In certain industrial applications, the separation process is vital. This is especially true when high purity chitosan nanoparticles are needed and in low volume applications.

The present invention illustrates the use of chitosan nanoparticles along with its process solution for large volume industrial applications such as surface decontamination, food, and agriculture. The present method provides nanoparticle preparation without separation or purification processes which dramatically reduces the production cost and results in improved performance for high volume applications.

In the ionotropic gelation method, the protonated amine group of chitosan interacts with an anion of the gelation agent via ionic interaction to form nanoparticles under the acidic conditions. The attraction between the protonated amine group of chitosan and the anion of the gelation agent is a reversible, physical crosslinking by electrostatics interaction, not by a strong chemical bonding or chemical modification. The formed nanoparticles with positive zeta potential are stable in the ionic process solution for an extended time period. During the various applications, the electrostatic interaction can be exchanged by stronger negatively charged or high affinity compounds or with a halogen molecule. This allows for the effective delivery of chitosan nanoparticles with or without a halogen for their applications with strong adhesion or immobilization properties against negatively charged surfaces in an aqueous process solution. This unique property of chitosan nanoparticle composite in the presence of process solution with a positive zeta potential provides a great opportunity to bypass the separation process during manufacturing. This method of preparation can be desirable for certain applications where the presence of process solution is not a concern and/or presence of a process solution adds value to the application.

### BRIEF DESCRIPTION

Various embodiments of the present disclosure relate to the methods of producing economical chitosan nanoparticle composite and methods and systems for removing surface contaminants, wound healing and agricultural applications.

A first embodiment of the present disclosure provides a method for producing chitosan nanoparticle composite. The method comprises converting chitosan or derivatives thereof to nanoparticles by an ionotropic gelation process at pH of less than about 6.5 by using an organic acid, gelation agent, and a nonionic surfactant in an aqueous solution. The prepared nanoparticle composite can be used without any further separation or purification procedures.

In various embodiments, the acidic chitosan aqueous solution may be prepared by using an organic acid which may include acetic acid or citric acid or lactic acid or malic acid or these acids in combination. Secondly, a nonionic surfactant is added to chitosan solution. In certain embodiments, sources of a nonionic surfactant may include the source of Polysorbate 80 (Tween 80). Lastly, the gelation agent is added to the acidic chitosan solution which containing a nonionic surfactant under continuous agitation. In various embodiments, the process include the gelation agent is added drop-wise to chitosan solution containing a nonionic surfactant using a magnetic stir. In various embodiments, sources of ionotropic gelation agent may include sources of Sodium Tripolyphosphate (STPP), Sodium Dodecyl Sulfate (SDS), and Sodium Sulfate (SS) and combinations of any thereof.

In certain embodiments, the described method can further comprise loading the chitosan nanoparticles in the process solution with an active halogen using a source of soluble halogen at pH higher than about 4.0. In various embodiments, sources of active halogen may include sources of active chlorine selected from the group consisting of trichloroisocyanuric acid (“TCCA”), sodium dichloroisocyanuriate, sodium hypochlorite, calcium hypochlorite, hypochlorous acid, and combinations of any thereof. In various embodiments, sources of active halogen may include sources of soluble iodine (I2) or bromine (Br2).

Still further embodiments of the present disclosure provide methods for producing chitosan nanoparticle composite in the presence of process solution and application methods for improved disinfectant and fungicidal properties. The chitosan nanoparticle composite can be used to clean various contaminated surfaces including household hard surfaces such as hard wood, tile, bathroom and kitchen and plumbing.

In certain embodiments, this nanoparticle composite with incorporated nontoxic process solution can be directly used for disinfecting food production surfaces, and food surfaces including poultry, ready-to-eat meat, dairy products and fresh produces. In certain embodiments this nanoparticle composite can be used to prevent wound infections. Moreover this nanocomposite delivery of chitosan can be very effective for agricultural applications since the approach allows for efficient production of large volume preparations.

### DESCRIPTION OF THE DRAWINGS

The various embodiments described herein may be better understood by considering the following description in conjunction with the accompanying drawings, wherein:

FIG. 1 illustrates the method of producing chitosan nanoparticles according to the present disclosure;

FIG. 2 illustrates the method of producing halogenated chitosan nanoparticles according to the present disclosure;

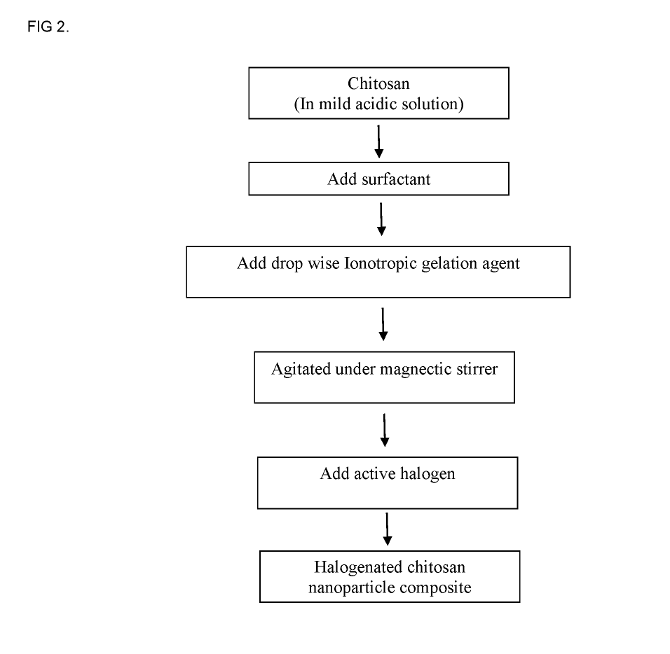

### DETAILED DESCRIPTION

Embodiments of the present disclosure provide a method of producing economical chitosan nanoparticle composites. The present method may display a cost effective production of nanoparticles in composites and reduces technical difficulties in the manufacturing processes. The present disclosure defers from the traditional methods of nanoparticle preparation by eliminating the separation and purification procedures. The traditional gelation method includes adding gelation agent into the acidic chitosan solution, followed by separation and purification procedures to concentrate and purify the nanoparticles. In the use of traditional method, a centrifugation process is commonly used to separate the nanoparticles from the process solution, followed by a purification process. The purification process includes sequential rinsing with distilled water or specific chemicals. This rinsing procedure requires subsequent centrifugation to separate out the rinsing solution and nanoparticles. The separation and purification procedures are critical for certain applications especially for biomedical applications which require highly pure chitosan nanoparticles. The present disclosure provides a simplified, economical method for preparing chitosan nanoparticles without a separation procedure for certain applications. Further, the presence of process solution especially the presence of gelation agent prevents the aggregation of nanoparticles in the nanocomposite. Thus allows effective use of the nanoparticles for extended time.

The method of preparation of chitosan nanoparticle composite in the present disclosure may comprise an active halogen source for certain applications. The chitosan nanoparticle composite or halogenated chitosan nanoparticle composite may be used to clean surfaces in order to remove biological contaminants, such as viral, bacterial, microbial, and/or fungal contaminants. Based on these various embodiments, the resulting chitosan nanoparticles displays lower production costs with improved consistent performance in removing biological contaminants from various surfaces with minimal halogen usage. This present disclosure provides the method of using halogens in its N-halamine active form for improved performance in removing biological contaminants as well as cleaning surfaces. Further, this present disclosure provides the method of cleaning surfaces with reduced wash water usage due to the presence of minimal amount of halogens in its active stable form.

As generally used herein, the terms “include” and “have” mean “comprising”.

As generally used herein, the term “about” refers to an acceptable degree of error for the quantity measured, given the nature or precision of the measurements. Typical exemplary degrees of error may be within 20%, 10%, or 5% of a given value or range of values. Alternatively, and particularly in biological systems, the term “about” may mean values that are within an order of magnitude, potentially within 5-fold or 2-fold of a given value.

All numerical quantities stated herein are approximate unless stated otherwise, meaning that the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as precisely as possible.

All numerical ranges stated herein include all sub-ranges subsumed therein. For example, a range of “1 to 10” is intended to include all sub-ranges between and including the recited minimum value of 1 and the recited maximum value of 10. Any maximum numerical limitation recited herein is intended to include all lower numerical limitations. Any minimum numerical limitation recited herein is intended to include all higher numerical limitations.

As used herein, “to reduce contaminants” and “reducing contaminants” and refer to disarming one or more contaminants in the surface, whether by physically or chemically killing, removing, reducing, or inactivating the contaminants or otherwise rendering the one or more contaminants harmless.

In the following description, certain details are set forth to provide a thorough understanding of various embodiments of the apparatuses and/or methods described herein. However, a person having ordinary skill in the art will understand that the various embodiments described herein may be practiced without these details. In other instances, well-known structures and methods associated with the apparatuses and/or methods described herein may not be shown or described in detail to avoid unnecessarily obscuring descriptions of the embodiments described herein.

This disclosure describes various features, aspects, and advantages of various embodiments of surface decontaminating systems as well as methods of making and using the same. It is understood, however, that this disclosure embraces numerous alternative embodiments that may be accomplished by combining any of the various features, aspects, and advantages of the various embodiments described herein in any combination or sub-combination that one of ordinary skill in the art may find useful.

Any patent, publication, or other disclosure material, in whole or in part, recited herein is incorporated by reference herein but only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used herein, the term “chitin” refers to a polymer of β-1,4-(2-deoxy-2-acetamidoglucose) that may be extracted from the exoskeletons of insects and arthropods, such as crabs, lobsters and shrimps, and cell walls of fungi and yeast. As used herein, the term “chitosan” refers to derivative of chitin having a polymeric structure comprising 2-deoxy-2-acetamidoglucose monomers and 2-deoxy-2-aminoglucose monomers and typically comprises greater than 70% deacetylated 2-deoxy-2-aminoglucose monomer units. Chitosan may be formed from chitin by hydrolyzing a portion (i.e., greater than 70%) of the 2-deoxy-2-acetamidoglucose monomeric units to 2-deoxy-2-aminoglucose monomeric units. Chitosan may be fully or partially deacetylated chitin. Chitosan comprises a polymer backbone comprising hydroxyl groups and amine groups. Chitosan may be soluble in aqueous acidic (pH <6.0) solutions. As used herein, the term “partially deacetylated chitosan” or “partially deacetylated chitin” refer to a polymeric structure having 2-deoxy-2-acetamidoglucose monomers and 2-deoxy-2-aminoglucose monomers and having a percent deacetylated units as described herein, for example, from about 5% up to 70% deacetylated 2-deoxy-2-aminoglucose monomer units, or in some embodiments from about 5% to 60% deacetylated 2-deoxy-2-aminoglucose monomer units. As used herein, the term “chitosan-based material” refers to the product formed by contacting chitosan or chitin according to the methods described herein.

The chitosan nanoparticles may be prepared from the chitosan or derivatives which may have a molecular weight in the range of from 5,000 Daltons to two million Daltons, such as from 50,000 Daltons to one million Daltons, or such as from 100,000 Daltons to 900,000 Daltons. The source of chitosan may have a percentage of deacetylation from 40% to 100%, such as from 60% to 95%, or from 70% to 90%. In certain embodiments, the nanoparticle composite may be formed from the chitosan or derivative thereof may comprise a powder having a U.S. standard mesh size from 30 mesh to 230 mesh.

As used herein, the term “active halogen” refers to compounds having active forms of an element of the group 17 column of the periodic table (i.e., F, Cl, Br, and I), for example active halogen includes compounds having a molecular formula of X₂, where X is one of F, Cl, Br, or I, compounds, or having a formula, such as OI⁻, I₃⁻, OBr⁻, or OC1⁻. Examples of active halogens include, but not limited to, Cl₂ and Br₂. Halogen (X₂) producing compounds include compounds that release a halogen into aqueous systems. Active halogen, as used herein, corresponds to a active species consisting of a single type of halogen (i.e., only I, only Cl, or only Br). As used herein, the term “iodine” means molecular iodine with a formula I₂. As used herein, the term “halide” refers to the anionic form of a halogen atom, represented as X⁻. Examples of halide ions include chloride (Cl⁻), bromide (Br⁻) or iodide (I⁻).

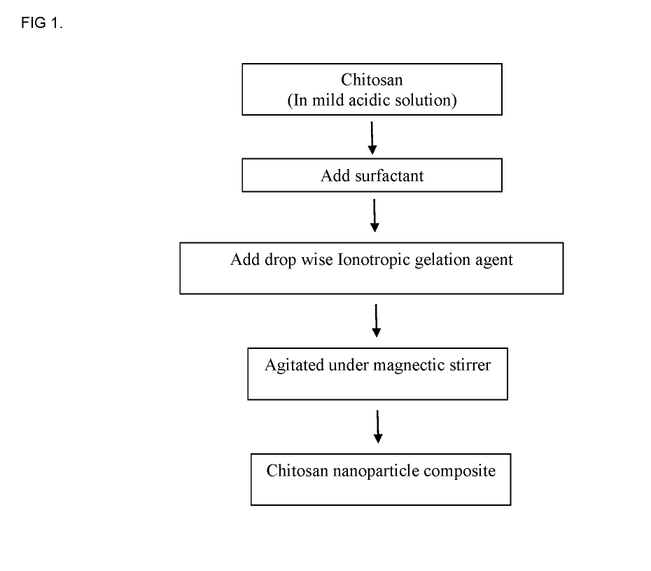

The conventional chitosan nanoparticle preparatory methods include a separation and purification process to isolate the nanoparticles. This is a major drawback in high volume applications. FIG. 1, illustrates the present method of production of chitosan nanoparticle composite. The present method is comprised of an acid chitosan aqueous solution, containing a nonionic surfactant which is then treated with a gelation agent to perform ionotropic gelation process under agitation in order to form chitosan nanoparticle composite. The nanoparticle composite may be used in various applications without a separation or purification process.

As used herein, the term “Ionotropic gelation” refers the process of forming chitosan nanoparticles using ionic interactions. The protonated chitosan amine groups interact with specific anions of the gelation agents such as sodium tripolyphosphate (STPP), sodium dodecyl sulfate (SDS), and sodium sulfate (SS). This process is facilitated by inter- and intra-molecular cross linkages by the anions of the gelation agents. It is a reversible, physical cross linking by electrostatic interaction which may be readily modified. The chitosan nanoparticles in the presence of a gelation agent results in a positive zeta potential that is well suited for certain applications since the interaction can be altered with stronger negatively charged components or other stronger affinity chemicals. Although not based on any specific theory, it can be expected that the potential activities of chitosan nanoparticles in the composite are due to ready available positive charge.

As used herein, the term “separation” refers as any kind of separation process to separate chitosan nanoparticles from the process solution. The separation includes particle separation methods such as centrifugation, ultracentrifugation, membrane filtration, ultrafiltration, sedimentation, dialysis, electro dialysis, drying, freeze-drying, reverse osmosis, elutriation, electrophoresis, electrolysis, electrostatic precipitation, flotation, screening, magnetic separation, and filed flow fractionation. The term further includes all other physical and chemical separation methods used in the industrial process including crystallization, partition, and precipitation. The term further includes various combined methods such as chromatography, ion exchange, adsorption, distillation, extraction, equilibrium separation, form fractionation, sublimation, evaporation, drying, flocculation, exclusion, sieving, magnetic separation, decantation and clathration.

As used herein, the term “purification” refers as the process further purify the nanoparticles after the separation process. The purification may include sequential rinsing of nanoparticles with deionized water or distilled water or with other chemical compounds followed by the methods described as “separation”.

The present disclosure provides a novel inventive approach for producing chitosan nanoparticle composite. The chitosan nanoparticle composite produced according to the various embodiments herein provide improved biological decontamination, wound healing and growth promotion compared to conventional chitosan applications developed for these purposes. The chitosan nanoparticle composite is an ideal approach for these applications due to its effective size dependent activity. Further these nontoxic nanoparticle composite can provide prolonged residual effects without affecting the overall quality of the potential applicable biological/non-biological surfaces.

The size and zeta potential (ZP) are the major features of the chitosan nanoparticles for determining the effectiveness in terms of decontamination, adherent properties and cellular responses. As used herein, the term chitosan nanoparticles refers to sizes ranging from diameter 10-2000 nm. As used in here the term refer zeta potential (ZP), refers to the overall charge that nanoparticles acquire during the composition process that can be determined by the zetasizer nano instrument. The present chitosan nanoparticle composite is comprised of nanoparticles with ZP ranging from 1-200 mV.

In certain embodiments, the surface cleaning material may comprise chitosan nanoparticles. In certain embodiments, the nanoparticles of chitosan or derivative thereof may comprise a nanoparticle having a size from 10 nanometers to 100 nanometers. In certain embodiments, the nanoparticles of chitosan or derivative thereof may comprise a nanoparticle having a size from 100 nanometers to 450 nanometers. In certain embodiments, the nanoparticles of chitosan or derivative thereof may comprise a nanoparticle having a size from 10 nanometers to 2000 nanometers. The required nanoparticles size can be designed by adjusting the ratio of chitosan versus gelation agent in the composite.

In certain embodiments, the initial chitosan solution may comprise an organic acid in order to maintain a pH of around, but not exceeding, 6.5. In certain embodiments, the organic acid may contain the group of selected acetic, citric, lactic or malic acid. In certain embodiments, the organic acid is an aqueous acetic acid with percentage ranging from 0.01 to 2%. In certain embodiments, the percentage of initial chitosan solution may comprise from 0.01-0.5%. In certain embodiments, the percentage of starting chitosan solution may comprise the 0.25% (w/w). In certain embodiments, the initial acidic chitosan solution may comprise a nonionic surfactant. In certain embodiments, the nonionic surfactant may comprise polysorbate 80 (tween 80) at percentage ranging 0.01-1%. In certain embodiments, the acid chitosan solution may comprise polysorbate 80, at 1%.

In certain embodiments, the chitosan nanoparticles may be formed by the drop wise addition of gelation agent to the acidic chitosan solution containing surfactant under magnetic stirring. In certain embodiments, The gelation agent may be comprised the group of sodium tripolyphosphate (STPP), Sodium dodecyl Sulphate (SDS) or Sodium Sulphate (SS). In certain embodiments, the gelation agent may be comprised of STPP at percentage ranging 1 to 20%. In certain embodiments, the STPP percentage may be 10%. In certain embodiments, the gelation agent may be comprised of SDS ranging from 1-20%. In certain embodiments, the gelation agent may be comprised of SS ranging from 1-20%.

In certain embodiments, the ratio of % of gelation agent and % of chitosan in the process composition may range from 0.1:2 to 1:2. In certain embodiments, the % of gelation agent and % of chitosan may be in ratios of 0.6: 1, 0.5:1 or 0.4:1. The ratios play a major role in determining the size of the nanoparticle in the composite. The increasing concentration of the gelation agent is reducing the size of the chitosan nanoparticles.

In certain embodiments, the mechanical agitation may provide for nanoparticle formation. The mechanical agitation may be performed at 100, 200, 300, 400, 500 rpm. In certain embodiments, the magnetic stirring may be performed at 500 rpm. The mechanical agitation may be performed for 1 min to 3 h. In certain embodiments, the mechanical agitation may perform for 2 h.

The method of preparing chitosan nanoparticle composite in the various embodiments of the present disclosure includes the formation of chitosan nanoparticles by ionotropic gelation without any further separation or purification process prior to application. The method includes the acidic chitosan solution containing a nonionic surfactant treated with gelation agent under agitation. The prepared nanocomposite may be applied in the presence of a process solution which may include water, nonionic surfactant and the gelation agent.

In certain embodiments, the present disclosure includes an active halogen molecule loaded on the chitosan nanoparticles. The halogen incorporation can be performed by adding a soluble halogen source to the prepared nanoparticle composite. Without binding any specific theory, it can be summarized that the halogen can bind to the amine group of the chitosan nanoparticles to form N-halamine. The N-halamine form of chitosan has been demonstrated for effective antimicrobial and antifungal properties. A method for forming the chitosan-halogen complex may involve contacting the chitosan nanoparticle composite with a halogenating agent. As a result of the reaction of the chitosan nanoparticle composite with the halogenating agent, at least a portion of the chitosan 2-deoxy-2-aminoglucose monomeric units may be converted to 2-mono aminoglucose monomeric units and/or 2,2-dihalo aminoglucose monomeric units to yield the chitosan-halogen complex. The halogenating agent may be comprised of any agent containing a halogen, such as chlorine, bromine, and iodine, capable of donating a halogen atom. The halogenating agent may be at least one of sodium hypochlorite, calcium hypochlorite, chlorine, bromine, iodine, aqueous chlorine solutions, aqueous bromine solutions, aqueous iodine solutions, N-chlorosuccinimide, sodium hypobromite, pyridinium bromide perbromide, N-bromosuccinimide, and/chloramine-T. Other suitable halogenating agents will be readily apparent to those skilled in the art.

The present disclosure provides the method of loading active halogen to the chitosan nanoparticles which dramatically reduces the amount of halogen needed for surface cleaning applications. Without binding any particular theory, it is believed that the present disclosure provides dual mechanistic activity against contaminants as initial biostatic performance by oxidative potential of active halogen followed with the residual effects of positively charged chitosan nanoparticles in the presence of process solution. The active halogen attached on the chitosan amine group is stable and effective against contaminants, in a manner similar to its original form. Additionally, the present disclosure results in the added benefit of reducing the volume of wash water for effective cleaning purposes due to limited amount halogen usage.

In certain embodiments, the surface decontaminating material may be comprised of a nanoparticle composite containing a chitosan-halogen complex. The halogen may be encapsulated in the lattice matrix of the nanoparticles of chitosan or derivative thereof. The nanoparticles of chitosan-halogen complex may be selected from the group consisting of a nanoparticles of chitosan-chlorine complex, chitosan-bromine complex, a chitosan-iodine complex, and/any combination thereof. Without wishing to be bound to any particular theory, it is believed that the halogen in the nanoparticles of chitosan-halogen complex is readily available in free from. The nanoparticles of chitosan-halogen complex comprises the association of the halogen and nanoparticles of chitosan or derivatives thereof. The nanoparticles of chitosan-halogen complex generally involve a reversible association of molecules, atoms, or ions through weak chemical bonds. In at least one embodiment, the nanoparticles of chitosan-halogen complex may comprise a chlorinated chitosan. The chlorine molecules in the nanoparticles of chitosan-halogen complex may be readily available as free chlorine.

In certain embodiments, the nanoparticles of chitosan-halogen complex may be comprised of an iodinated chitosan nanoparticle. The nanoparticle of chitosan-iodine complex may include iodine and/or iodide complexed to the nanoparticle of chitosan or derivative thereof. Suitable iodides include, but are not limited to, iodine-iodide complexes of the form (cation)⁺(I₃)⁻, wherein the cation is a cationic small molecule, such as a metal ion, e.g., potassium or sodium ions, or a cationic group attached to the chitosan, and I₃ is the tri-iodide anion. Examples of chitosan-iodine complexes are described in U.S. Pat. Nos. 4,275,194 to Kato et al., 5,204,452 to Dingilian, et al., 5,362,717 to Dingilian, et al., 5,336,415 to Deans, 5,538,955 to Rosa et al., and 6,521,243 to Hassan.

In at least one embodiment, the nanoparticles of chitosan-iodine complex may be comprised up to 50% of bound iodine by weight of the chitosan. In at least one embodiment, the nanoparticles of chitosan-iodine complex may comprise up to 60-70% of bound iodine by weight of the chitosan. The concentration of the iodine may depend on the components of the composition. In certain embodiments, the concentration of the iodine may be the range of at least 0.05% by weight, at least 0-1%, and at least 0.5%, and ranging upward to 1%, 2%, 3%, 4%, 5% or more. Higher concentrations may be used when the iodine is stable against aggregation and evaporation during the product's shelf life.

In various embodiments, the surface decontaminating material may be comprised a chitosan nanoparticle composite in the presence of process solution with or without containing a chitosan-halogen complex in order to provide effective surface decontamination of pathogenic microbial and fungal contaminants. Various surfaces such as hard surfaces, including tile, hard wood and laminate floor, plumbing fixtures, and kitchen utensils can be decontaminated by the present nanoparticle composite. Further, this nontoxic nanoparticle composite can be utilized in the food industry for cleaning food production surfaces, as well as decontaminating food surfaces such as poultry, meat, and ready-eat products, fresh produces including fruits and vegetables.

In various embodiments, the surface decontaminating material may be comprised of chitosan nanoparticle composite in the presence of process solution with or without containing chitosan-halogen complex that can be applied as aerosol spray with adjustable nozzle size for specific applications. In certain embodiments, the nanoparticle composite is sprayed, followed by wiping with suitable material. In other embodiments, the nanoparticles composite is loaded to an absorbable pad or sponge, then applied as surface wipe for decontamination purposes.

In certain embodiments, the present disclosure provides ideal delivery systems for wound healing applications. The chitosan and chitosan formulations, complexes, and derivatives with other substances have been researched extensively. More specifically the wound healing properties of chitosan with Iodine (12) has been demonstrated by U.S. Pat. No. 4,275,194 A. The wound healing mainly focuses on prevention of infection, maintenance of moist environment, and rapid healing with minimal scar formation. When using traditional chitosan formulations for wound dressing there is possible air or fluid pocket formation due to its complex structure and strong adherent properties. In the traditional formulations the limited active contact surface may mask the activity of the chitosan. The present disclosure provides greater wound healing benefits with highly effective nano-sized particles in the presence of safe gelation agent with uniform applications and preventing air or fluid pocket formation.

In certain embodiments, the present disclosure provides effective use of chitosan in agriculture. The chitosan and its derivatives have been found to have several agricultural benefits including, growth promotion, enhance immune responses, improve crop protection and soil fertility. The size of the chitosan particles is critical for agricultural applications, especially for crop protection. The chitosan oligosaccharides with defined size are widely used in agriculture. The acid hydrolysis of chitosan at high temperature is a common method for preparing large amount of glucosamine for agriculture applications. Controlling the degree of degradation of the chitosan polymer chain is very difficult during this chemical process. The over degradation of the chitosan polymer chain may yield shorter chain length which cannot be used for specific agricultural applications. The present disclosure provides the method of preparing chitosan nanoparticles with defined size for agricultural applications especially when applied as elicitors. Moreover, this method is more economical and provides room for higher production volume without any difficult nanoparticle separation process. By adjusting the concentrations of various process solutions, the nanoparticle with defined sizes can be prepared and stored for extended time without any aggregation. The foliar application of this nanocomposite can reduce the water loss by transpiration. The elimination of separation process of production of these nanoparticles is vital for large volume agricultural applications. Further, the selected gelation agent presence in the chitosan nanoparticle composite may beneficial for certain agricultural applications especially where nutrient supplement is required.

The present disclosure provides novel and inventive methods for producing a chitosan nanoparticle composite. The method of preparation without separation of the process solution is beneficial in high volume applications. The presence of process solution in the nanoparticle composite may add additional benefits. The gelation agents STPP, SDS, SS and polysorbate 80 are safe, nontoxic compounds sometimes used as food additives. The SS is used for various wound healing solutions for topical applications. Further the STPP can act as a phosphate supplemental agent to the crop production systems.

These and other features of the various embodiments of the present disclosure will become more apparent upon consideration of the following examples. The various embodiments of this disclosure described in the following examples are not to be considered as limiting the invention to their details.

### EXAMPLES

As generally used herein, the terms “ND” refers to not detectable or below the detection limit and “NA” refers to not applicable

For the present examples, the industrial chitosan (Low Molecular Weight: 0.8-1.5 kDa, % Degree of Deacetylation: is obtained from Bio21, Ltd, Thailand. The chemicals were obtained from the following sources, although other sources are possible. The acetic acid 99.8% from Acros Organics, Fair Lawn, N.J. The Sodium Trypolyphophate (85% tech) was obtained from Acros Organics, Fair Lawn, N.J. The Sodium Lauryl Sulfate (CAS 151-21-3) was obtained from Fisher Scientific, N.J. The tween 80, non-animal source (P6224) was obtained from Sigma-Aldrich. Iodine crystals USP (CAS# 7553-56-2) were obtained from Deep Water Chemicals, Subsidiary of Tomen America Inc., Woodward, Okla.

The chitosan nanoparticle composites were prepared using two different ionotropic gelation agents with different compositions. The chitosan solution was prepared by adding acetic acid, and subsequently adding tween 80 during magnetic stirring. The ionotropic gelation agent sodium tripolyphosphate or sodium lauryl sulfate or sodium sulfate was added drop wise to the chitosan solution containing tween 80, under magnetic stirring (350 rpm) and continued for 2 h. For halogenated chitosan nanoparticle composite preparation, the iodine crystals were added to chitosan nanoparticle composite under magnetic stirring.

### Example 1

### Comparison of Conventional and Present Method of Producing Chitosan Nanoparticle and Chitosan Nanoparticle Composite

As shown in Table 1, the present method is lacking the step 4 and 5. These procedures, steps 4 and 5 are labor oriented time consuming and creates practical difficulties in high volume industrial production.

TABLE 1
The method of producing conventional chitosan nanoparticles
and the present chitosan nanoparticle composite

| Process | | |
|---|---|---|
| of Chitosan | | |
| Nanoparticles | Conventional Method | Present Method |
| Step 1 | Acidic Chitosan solution | Acidic Chitosan solution |
| Step 2 | Adding a nonionic | Adding a nonionic |
| | surfactant | surfactant |
| Step 3 | Drop wise adding gelation | Drop wise adding gelation |
| | agent to perform | agent to perform |
| | “Ionotropic gelation” to | “Ionotropic gelation” to |
| | form nanoparticles under | form nanoparticles under |
| | agitation | agitation |
| Step 4 | “Separation” of | No “Separation” process |
| | nanoparticles by | Nanoparticles is kept in the |
| | centrifugation (Discard | process solution |
| | process solution) | |
| Step 5 | “Purification” of | No “Purification” process |
| | nanoparticles by sequential | |
| | rinsing followed by | |
| | centrifugation | |
| Step 6 | Applied as nanoparticles | Applied as nanoparticle |
| | with or without other | composite |
| | additives | |

### Example 2

The particle size distribution of chitosan nanoparticles in the nanoparticle composite (The nanoparticle composite comprising: Chitosan—0.25%, Acetic acid—2%, Tween 80 (Polysorbate 80)—1%, Sodium Tripolyphosphat—0.5%, Water—96.25%) has been evaluated using Master sizer v3.40 (Malvern Instruments Ltd.). The analysis provided by Desert Research Institute (DRI), Reno, Nev.

TABLE 2
Particle size distribution of chitosan nanoparticle in the nanocomposite

| Size (μm) | % Number In |
|---|---|
| 0.0100 | 53.57 |
| 0.500 | 35.23 |
| 1.00 | 7.24 |
| 1.50 | 2.21 |
| 2.00 | 1.25 |
| 3.00 | 0.31 |
| 4.00 | 0.10 |
| 5.00 | 0.04 |
| 6.00 | 0.02 |
| 7.00 | 0.01 |
| 8.00 | 0.01 |
| 9.00 | 0.00 |

Data: Master sizer v3.40 (Malvern Instruments Ltd.), Particle Refractive Index 1.335, Particle Absorption Index 0.001, Dispersant Name Water, Dispersant Refractive Index 1.330, Scattering Model Mie, Analysis Model General Purpose, Weighted Residual 0.39%, Laser Obscuration 16.73%, Concentration 0.2200%, Span 1.594, Uniformity 0.532, Specific Surface Area 8325 m²/kg, D [3|2] 2.40 μm, D [4|3] 9.72 μm, Dn (10) 0.290 μm, Dn (50) 0.477 μm, Dn (90) 1.05 μm.

### Example 3

TABLE 2
Nanoparticle composite for Kitchen utensils

| Component | Weight Percentage |
|---|---|
| De-ionized water | 98.3 |
| Chitosan | 0.2 |
| Acetic acid | 0.1 |
| Tween 80 | 0.9 |
| Sodium Lauryl Sulfate | 0.5 |

### Example 4

TABLE 3
Nanoparticle composite for food preservation

| Component | Weight Percentage |
|---|---|
| De-ionized water | 98.3 |
| Chitosan | 0.2 |
| Acetic acid | 0.1 |
| Tween 80 | 0.9 |
| Sodium Tripolyphosphate | 0.5 |

### Example 5

TABLE 3
Nanoparticle composite for wound healing

| Component | Weight Percentage |
|---|---|
| De-ionized water | 98.2 |
| Chitosan | 0.2 |
| Acetic acid | 0.1 |
| Tween 80 | 0.2 |
| Sodium Sulfate | 0.5 |
| Iodine | 0.8 |

The invention claimed is:

1. A method for producing economical nanoparticle composite, comprising: converting chitosan or derivative thereof to nanoparticles by ionotropic gelation at pH less than about 6.5 and in the presence of a process solution ready for application without a separation process.

2. The method of claim 1, wherein the source of ionotropic gelation agent at least one of sodium tripolyphosphate (STPP), sodium dodecyl sulfate (SDS) and sodium sulfate (SS)

3. The method of claim 1, wherein the acidic source of organic acid selected from the group consisting of acetic acid, lactic acid, citric acid and malic acid or and combinations of any thereof.

4. The method of claim 1, wherein the chitosan or derivative thereof nanoparticle formation by ionotropic gelation process is facilitated by adding a nonionic surfactant.

5. The method of claim 4, wherein the nonionic surfactant is polysorbate 80 (tween 80).

6. The method of claim 1, wherein the chitosan or derivative thereof has a molecular weight from 5,000 Daltons to two million Daltons.

7. The method of claim 1, wherein the chitosan or derivative thereof has a percentage of deacetylation from 30% to 100%

8. The method of claim 1, wherein the nanoparticle composite thereof comprises chitosan nanoparticles having a size from 10 nanometers to 2000 nanometers.

9. The method claim 1, wherein the composition comprising anti-aggregation agent of chitosan nanoparticles

10. The method of claim 1, wherein the composition comprising a nontoxic disinfectant

11. The method of claim 1, wherein the composition comprising a nontoxic fungicide

12. The method of claim 1, wherein the composition comprising a nontoxic wound healing agent

13. The method of claim 1, wherein the composition comprising a nontoxic plant growth enhancer

14. The method of claim 13, wherein the composition comprising an elicitor

15. The method of claim 1, wherein the composition comprising a transpiration water loss reducing agent

16. The method of claim 1, further comprising of the method of producing halogenated chitosan nanoparticle composite by loading a soluble halogen source to a process comprising:
treating the chitosan or derivative thereof in an acidic aqueous solution containing a nonionic surfactant by at least one of gelation agent STPP, SDS, and SS under agitation to form chitosan nanoparticles; and adding solubilized halogen source to form N-halamine chitosan nanoparticle composite.

17. The method of claim 16, wherein the source of active halogen is at least one of chlorine (Cl.sub.2), iodine (I.sub.2) and bromine (Br.sub.2).

18. The method of claim 17, wherein the source of active halogen is a source of active chlorine selected from the group consisting of chlorine gas, trichloroisocyanuric acid (TCCA), sodium dichloroisocyanuriate, sodium hypochlorite, calcium hypochlorite, hypochlorous acid, and combinations of any thereof.

19. The method of claim 17, wherein the source of soluble active halogen halogen is a source of active iodine is selected from iodine crystals, and in situ preparation by iodide chlorine reaction and combination of any thereof.

20. The method of claim 17, wherein the composition comprising a halogenated disinfectant and fungicide
